(19) United States
(12) Patent Application Publication
(10) Pub. No.: US 2016/0030708 A1
(43) Pub. Date: Feb. 4, 2016

(54) RIFLED CATHETERS AND VASCULAR ACCESS SYSTEMS

(71) Applicant: AngioDynamics, Inc., Latham, NY (US)

(72) Inventors: Damon Casiello, Lowell, MA (US); Marissa Kewley, Medford, MA (US)

(73) Assignee: ANGIODYNAMICS, INC., Latham, NY (US)

(21) Appl. No.: 14/447,002

(22) Filed: Jul. 30, 2014

(51) Int. Cl. A61M 25/00; A61M 39/02; A61M 5/315; A61M 5/00

(52) U.S. Cl. CPC A61M 25/0043; A61M 5/007; A61M 39/0247; A61M 5/315; A61M 25/0097; A61M 2025/006; A61M 2039/0258

(57) ABSTRACT

A catheter having a shaft with a substantially circular profile and a catheter channel on the inner wall spiraling about the catheter longitudinal axis. In some embodiments, the catheter has multiple catheter channels spiraling about the catheter longitudinal axis. Certain embodiments include the catheter as part of a system, including an implantable port system with a rifled port outlet stem, and an infusion system with a rifled syringe distal component.

### FIELD OF THE INVENTION

The present invention relates generally to a catheter used for vascular access in a procedure that may require power injection of fluid (such as computerized tomography (CT) fluid), or power aspiration of fluid from a target site within the body. More specifically, the invention relates to an improved catheter, such as a peripherally inserted central catheter (PICC), a midline catheter, a central venous catheter, a dialysis catheter, or a port catheter having a rifled inner wall.

### BACKGROUND OF THE INVENTION

Medical professionals commonly use catheters for gaining prolonged access to an area within the body. Once the catheter tip is positioned at the target location, fluids such as CT fluid, or treatments such as antibiotics, chemotherapy, pain medicine, and nutrition can be administered. If the catheter tip is improperly positioned during insertion, various risks to the patient could potentially arise, including a fluid infusion that causes pain or injury to the patient, complications due to increased thrombosis rates, delays in therapy, catheter malfunction and additional costs to the patient or health care provider.

General standards for proper catheter placement depend on the type of catheter and the treatment being provided. For example, PICCs are commonly inserted into a brachial, cephalic or basilic vein in the arm and advanced through the venous system towards the superior vena cava (SVC). Current medical standards recommend that the distal tip of the catheter terminate in the lower ⅓ of the SVC, close to the junction of the SVC and the right atrium (RA).

Power injection of fluids is an automated form of delivering treatment to a patient via the catheter, substituting for the manual delivery of such treatment and fluids through a handheld device such as a syringe. Power injection usually involves the injection of fluid at electronically controlled and monitored flow rates and pressures. Flow rates and pressures are maintained by a computerized control unit automating the process. Power injection systems also have the capability to aspirate fluid from the body by applying a negative pressure to the catheter.

There are several problems that can occur during the process of power injecting fluid that may negatively affect catheter performance. Catheter whipping is an issue that has been observed during high flow rate power injection. Catheter whipping is a rapid side-to-side thrashing motion that occurs at the distal end of the catheter as fluid exits the catheter tip at a high velocity and flow rate. As seen in the heart diagram 10 shown in FIG. 1, the distal end 12 of the catheter 11 is positioned at the SVC/RA junction 21 of the heart 20. Fluid infused at a high flow rate will initiate the whipping motion at the distal end 12 of the catheter 11, causing the catheter to make contact with interior vessel walls and structures of the heart 20. If the whipping motion is violent, it can cause pain to the patient as well as damage to the heart, both being undesirable events.

Another issue that can arise from high pressure fluid injection is catheter dislodgement. As stated earlier, the preferred destination of a PICC catheter tip is the junction of the SVC and the RA. FIG. 2 shows examples of undesired areas in a patient 5 that the catheter can migrate to as a result of dislodgement due to excessive catheter movement. Excessive catheter tip movement and high flow rates and pressures could potentially cause the catheter to become dislodged from the SVC/RA junction and migrate up to the right internal jugular 23, or across to the brachiocephalic vein 22. A power injection while the catheter tip is positioned at one of these undesired locations could lead to serious injury to the patient.

There are also problems that can occur when power aspirating fluid at a high negative pressure. One issue is that the structural integrity of the catheter wall can be compromised. FIG. 3 shows the common problem of pinching when a prior art catheter 30 attempts to ramp-up aspiration at a high negative pressure. High negative pressures inside the catheter lumen can build a pressure differential that can cause the flexible catheter wall to collapse into the lumen, and can pinch the lumen shut. This pinching effect can add time and costs to the procedure and potential risk to the patient. For instance, many power injectors have a pressure alarm set to a pressure threshold that if reached, will automatically stop the injection or aspiration process to prevent exposing the patient to potentially harmful pressure levels. As the catheter wall begins to collapse, the lumen diameter decreases. A decrease in lumen diameter leads to a dramatic spike in lumen pressure levels, ultimately leading to a higher frequency in pressure alarms. Once the pressure alarm threshold is reached and aspiration is stopped, it may be necessary to restart all or part of the system and procedure initialization process. A further side effect of the catheter pinching shut is that it decreases the flow rate of aspiration, which may be a critical limitation for certain procedures, such as dialysis. In addition, repetitive bending and collapsing of the catheter wall into the lumen can compromise the integrity of the catheter shaft, and could produce a weak point on the shaft that could cause the catheter to burst during a subsequent high power injection.

### SUMMARY OF THE INVENTION

The present invention relates to a catheter used in procedures with power injection and power aspiration through the catheter.

In one embodiment, the invention is a catheter having a shaft with a proximal end, a distal end, an inner wall and a catheter longitudinal axis. The inner wall has a substantially circular profile and a first catheter channel spiraling about the catheter longitudinal axis.

In another embodiment, the invention is an implantable port system having the catheter described above and an implantable port having a reservoir, a septum fluidly sealing an opening to the reservoir, and an outlet stem in fluid communication with the reservoir. The outlet stem has a proximal end, a distal end, an inner wall and an outlet stem longitudinal axis. The inner wall of the outlet stem has a substantially circular profile and a first outlet stem channel spiraling about the outlet stem longitudinal axis. The catheter is configured to connect to the outlet stem.

In yet another embodiment, the invention is a catheter infusion system having a syringe and the catheter described above. The syringe has a syringe lumen that has a proximal and distal end, a plunger used to flush the syringe lumen and a distal syringe component. The distal component is made up of an inner wall and syringe longitudinal axis. The inner wall has a substantially circular profile and a first syringe channel spiraling about the syringe longitudinal axis. In this embodiment, the catheter above further has a luer at the proximal end and the syringe is configured to connect to the luer.

Embodiments according to the present invention can reduce the whipping action of the distal end of the catheter during high pressure and high flow rate fluid injection. Reduction of the whipping action of the catheter will minimize the potential of catheter malposition and damage to surrounding vessel walls and structures within the patient. In addition, catheter wall collapse during ramping up of aspiration will also be minimized, as vortical fluid flow into the distal end of the catheter works to promote and maintain catheter lumen patency.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 shows a diagram of the heart region of the body with the tip of a prior art catheter placed at the junction of the right atrium and superior vena cava;

FIG. 2 shows a diagram of the upper body including the heart and certain vascular portions;

FIG. 3 shows a perspective view of a prior art catheter pinching during high pressure aspiration;

FIG. 4 is a cross-sectional view of a catheter according to an embodiment of the invention, showing rifled channels and protrusions in the inner wall of the catheter;

FIG. 5 is a cross-sectional view of the catheter according to an embodiment of the invention, showing rifled channels and notches in the inner wall of the catheter;

FIG. 6 is a prospective view of a catheter according to an embodiment of the invention, showing vortical flow of fluid into the distal opening of the catheter during aspiration;

FIGS. 7A-7I are cross-sectional views of catheters according to exemplary embodiments of the invention; FIG. 7A shows a cross sectional view of a catheter with rectangular protrusions; FIG. 7B shows a cross sectional view of a catheter with rounded protrusions; FIG. 7C shows a cross sectional view of a catheter with triangular protrusions; FIG. 7D shows a cross sectional view of a catheter with trapezoidal protrusions; FIG. 7E shows a cross sectional view of a catheter with three protrusions; FIG. 7F shows a cross sectional view of a catheter with six protrusions; FIG. 7G shows a cross sectional view of a catheter with a substantially equivalent protrusion to channel ratio;

FIGS. 8A and 8B show a dialysis catheter according to an exemplary embodiment of the invention; FIG. 8A is a perspective partial cut-away view of the dialysis catheter, and FIG. 8B is a front profile view of the dialysis catheter lumen openings;

FIGS. 9A and 9B are cross-sectional views of catheters according to exemplary embodiments of the invention; FIG. 9A shows rifled channels at a first angle, and FIG. 9B shows rifled channels at a second angle;

FIGS. 10A and 10B are cross-sectional views of extrusion dies according to exemplary embodiments of the invention; FIG. 10A shows a cross-sectional view of an extrusion die configured to make a catheter with protrusions in the catheter inner wall; FIG. 10B shows a cross-sectional view of an extrusion die configured to make a catheter with notches in the catheter inner wall;

FIG. 11 is a perspective partial cut-away view of a port stem with a rifled lumen according to an embodiment of the invention;

FIG. 12 is a side view of a syringe according to an embodiment of the invention;

FIG. 13 is a partial cross-sectional view of the distal end of the syringe shown in FIG. 12, with a rifled lumen configuration; and

FIG. 14 is a cross-sectional view of a syringe insert with a rifled lumen according to an exemplary embodiment of the invention.

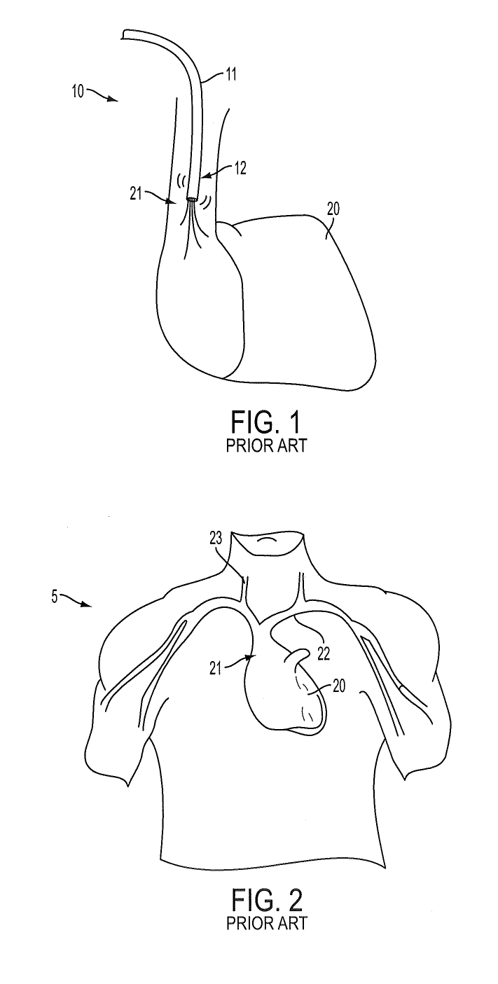

### DETAILED DESCRIPTION OF THE INVENTION

The present invention can be understood more readily by reference to the following detailed description, the examples included therein, and to the Figures and their following description. The drawings, which are not necessarily to scale, depict selected preferred embodiments and are not intended to limit the scope of the invention. The detailed description illustrates by way of example, not by way of limitation, the principles of the invention. The skilled artisan will readily appreciate that the devices and methods described herein are merely examples and that variations can be made without departing from the spirit and scope of the invention. It is also to be understood that the terminology used herein is for the purpose of describing particular embodiments only and is not intended to be limiting.

Referring now in detail to the drawings, in which like reference numerals indicate like parts or elements throughout the several views, in various embodiments, presented herein is an improved catheter and system for fluid infusion at high pressure and flow rate that reduces catheter whipping and migration, while improving catheter performance during ramp-up of catheter aspiration.

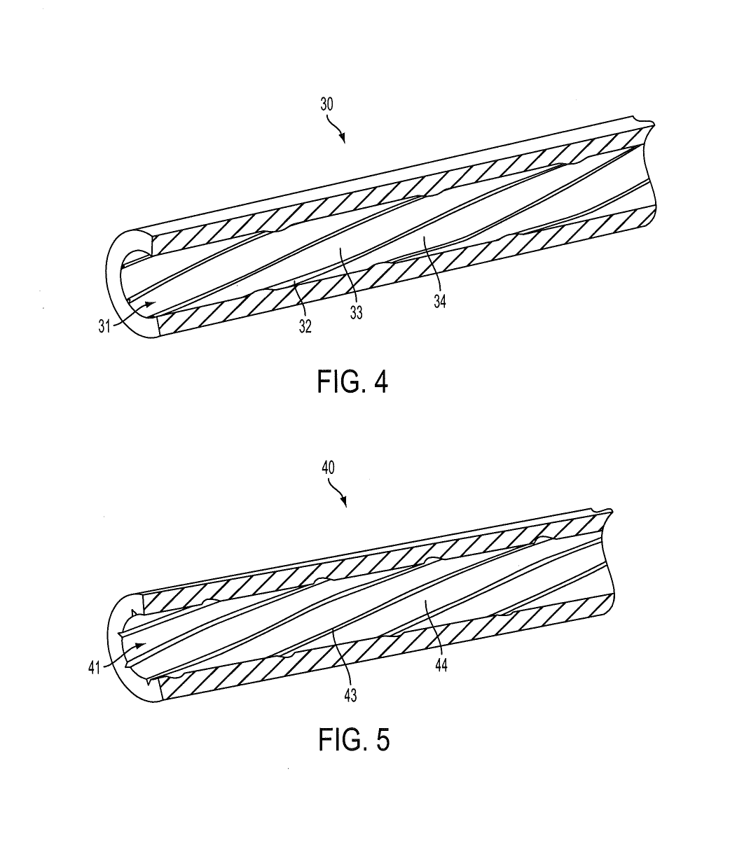

A catheter 30 according to an embodiment of the invention is shown in FIG. 4. The catheter 30 has a rifled catheter lumen 31 formed by channels 33 in the inner catheter wall 34 that spiral about the longitudinal axis of the catheter 30. The spiraling channel 33 on the inner catheter wall 34 creates a rifling effect, causing a vortical flow of fluid to move distally through the catheter during infusion. The spiral or vortical flow creates a centripetal force along the longitudinal axis of the catheter, and stabilizes the catheter at the target site during high flow rate infusions, reducing the likelihood of catheter whipping and migration. Further, turbulent flow that occurs in conventional catheters is minimized, as the fluid in catheters according to the present invention moves distally through the catheter in a more unitary fashion. Enhanced flow rates are due to the rifled channels helping to promote a more laminar flow of fluid, so that less pressure is required to achieve the same flow rate compared to a conventional catheter.

The rifling of the inner catheter wall 34 can be formed protrusions or notches. As shown in the embodiment of FIG. 4, the catheter 30 is formed with protrusions 32 on the inner catheter wall 34 so that fluid flow will spiral through the lumen as it travels from the proximal towards the distal end. Alternatively, as shown in the embodiment of FIG. 5, the channels 43 on the inner wall 44 of the catheter 40 can also be formed as notches 43 spiraling about the longitudinal axis of catheter lumen 41. In certain embodiments, the inner wall of the catheter can have a single channel on the inner catheter wall. Two or more channels can also be utilized. The number of channels may depend on a number of factors, including the properties of fluid being infused, the french size of the catheter, and the type of procedure being performed. These factors may also influence whether a notch or a protrusion is used, and what the width and height (or depth) of the channel will be. Alternative embodiments may use certain combinations of protrusions and notches within the same catheter lumen to create the rifled inner catheter wall.

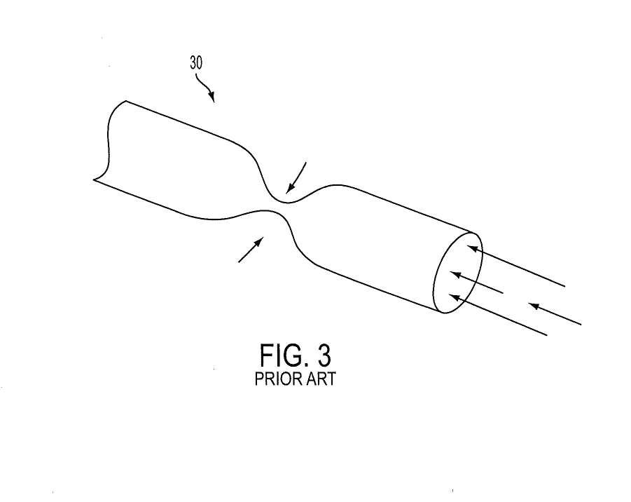

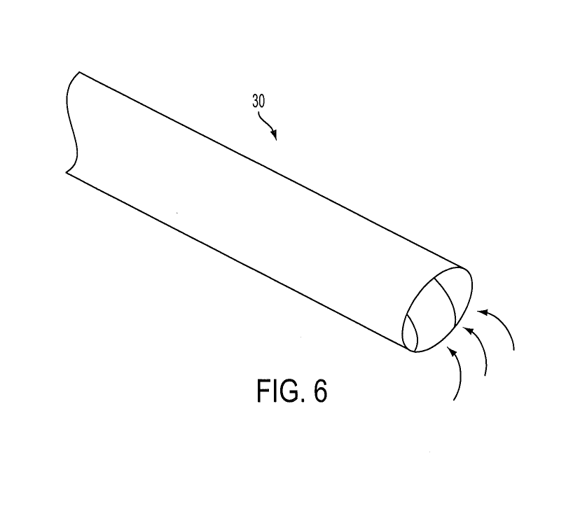

The advantages of the rifled catheter design during aspiration ramp-up are illustrated in FIG. 6. Before fluid enters the distal catheter tip, a negative pressure first begins to build in the catheter lumen, and a spiraling vortex of air or saline from a fluid lock is pulled through the lumen in a generally proximal direction. The air or saline will be followed by a more viscous and dense fluid, such as blood. The introduction of a more viscous material at the tip of the catheter commonly causes a conventional catheter to pinch as the negative pressure spikes in the lumen. The vortical and spiraling stream promotes lumen patency by redistributing the negative pressure from the linear stream (see FIG. 3) to a more vortical and laminar flow that is directed along the edges of the inner wall, promoting lumen patency. Since the fluid propagates though the catheter in a vortical path that generally matches the circular shape of the catheter structure, the fluid tends to curl into the diatal catheter opening, pushing up against the inner catheter wall, further minimizing pressure differential gaps that tend to cause the catheter wall to collapse. Further, the channels act as spacers that prevent the catheter lumen from completely closing, so that even if a catheter lumen starts to collapse, the geometry of the edges of the channels will continue to keep the lumen at least partially open, minimizing the chance for a dramatic spike in negative pressure and a pressure alarm.

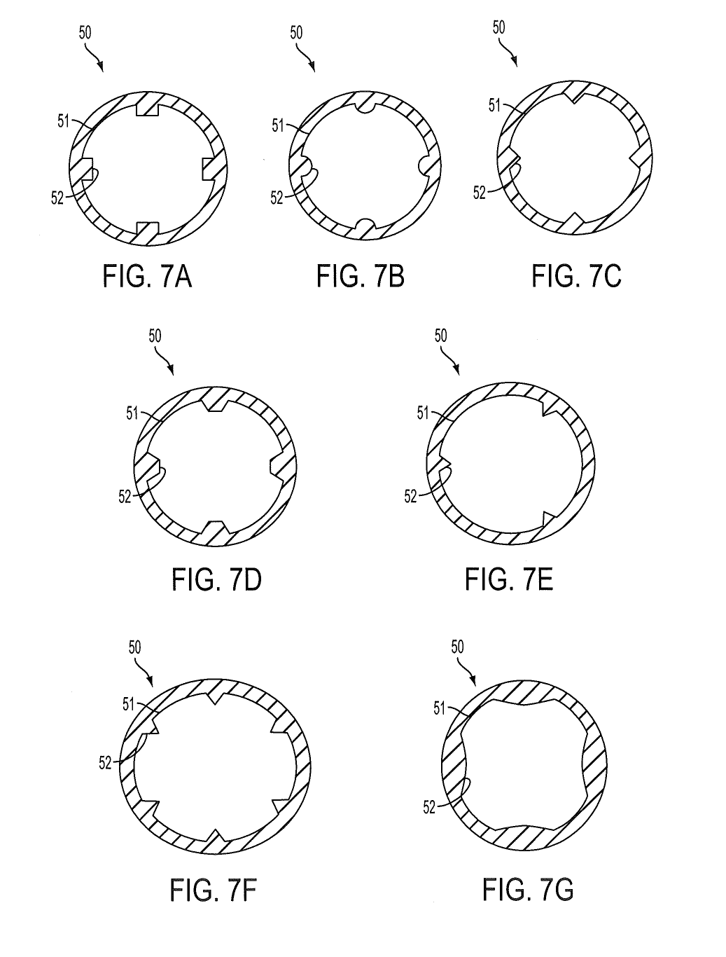

There are various geometries that can be used to create spiral flow in the catheter lumen to create catheter channels for vortical fluid flow. FIGS. 7A-7G demonstrate examples of these varieties. FIGS. 7A-7D demonstrate examples of shapes of protrusions 52 that can be used on the inner catheter wall 51. FIG. 7A shows a catheter cross-section 50 with protrusions 52 that are rectangular in shape. FIG. 7B shows a catheter cross-section 50 with protrusions 52 that are rounded in shape. FIG. 7C shows a catheter cross-section 50 with protrusions 52 that are triangular in shape. FIG. 7D shows a catheter cross-section 50 with protrusions 52 that are trapezoidal in shape. Although FIGS. 7A-7D show four protrusions, less protrusions, such as the three shown in FIG. 7E, or more protrusions, such as the six shown in FIG. 7F can be utilized. As few as one channel can be used to create the vortical flow. Further, one or more non-contiguous channels can disposed along one or more portions of the inner catheter wall along in a configuration sufficient for initiating the vortical flow described herein. Alternative widths of protrusions can also be utilized as shown in FIG. 7G, where the protrusion to channel ratio is substantially equivalent. Channels formed as notches can have similar geometric variability recessed into the inner catheter wall. Further, different combinations of geometries or channel sizes, such as a group of four protrusions that graduate in height for facilitating a more gradual initiation of vortical flow can be implemented. Tapered regions may result in a spiral having a variable distance from the longitudinal axis of the catheter lumen. For instance, a reverse taper region can include a transition zone which may for increases the distance of the spiral from the longitudinal axis in the proximal direction as the lumen size along the reverse taper increases. Various embodiments may increase or decrease the depth or height of the channel, or otherwise transition between two sets of geometric configurations, such as those disclosed herein. Constant diameter regions will often result in a spiral that can be described as more helical in nature.

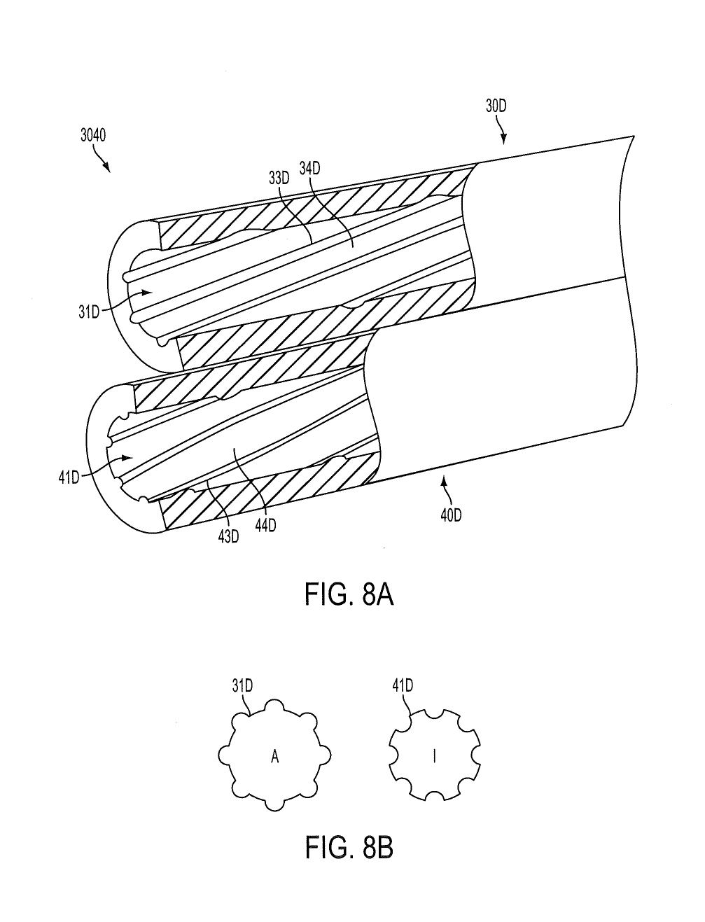

Dual lumen or multi-lumen catheters such as dialysis catheters may have dedicated lumens for aspiration and infusion, where the rifling patterns can be customized a particular function. For instance, a dialysis catheter 3040 as shown in FIG. 8A incorporates the features of the catheter 30 shown in FIG. 4 for the infusion lumen 31D, and the features of the catheter 40 shown in FIG. 5 for the aspiration lumen 40D. This may be beneficial for dialysis, where the arterial and venous lumens are dedicated for infusion and aspiration and terminate in a step tip or staggered tip configuration. Protrusions are advantageously used on the infusion lumen so that they can cut into the fluid path, initiating a tight vortical flow in the high flow rate fluid stream. Meanwhile, the aspiration lumen can advantageously feature notches, which in addition to the reasons provided above, will help ramp-up the uptake of fluid into the lumen by maximizing the profile of the aspiration lumen opening relative to the profile of the infusion lumen opening as shown in FIG. 8B. As more fluid is able to gather at the larger aspiration lumen 31D opening during aspiration ramp-up, fluid can enter and fill the lumen at a higher flow rate, maximizing lumen patency and minimizing the likelihood of catheter pinching. Apheresis ports may also feature a step or staggered tip catheter according to these embodiments.

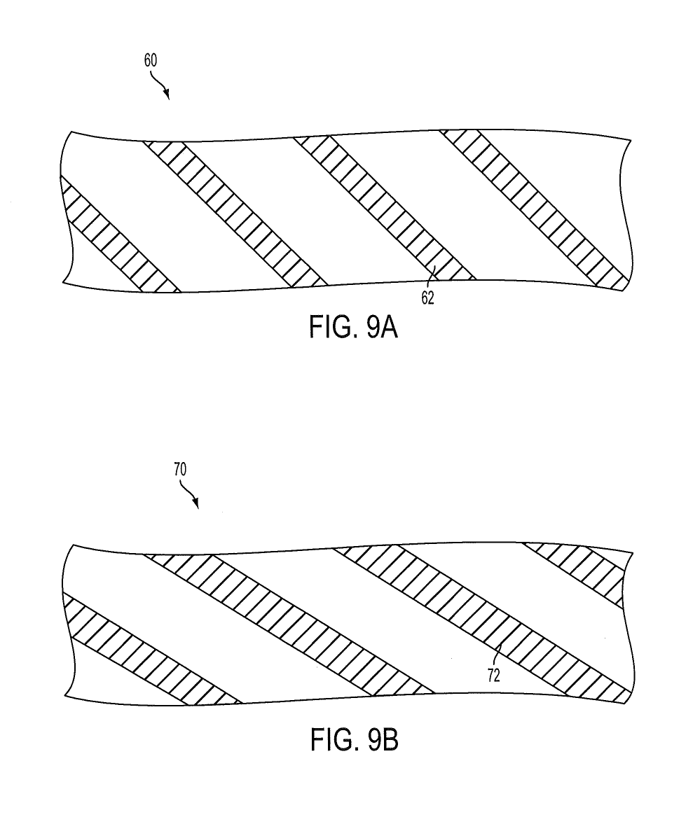

Optimal flow rates during power injection of fluids can be achieved by changing the angle of the rifles in the catheter which are responsible for creating the desired spiral flow pattern. As shown in FIGS. 9A and 9B, there can be various slopes at which the spirals 62/72 can be created in the catheter inner wall 60/70. Variation in slope may also exist along the length of any one particular lumen. For example, it may be desirable to start with a shallow slope or pitch of the catheter channel, so that the fluid can “catch” the channel and initiate vortical flow, then graduate to a steeper angle to optimize the vortical flow near the lumen opening. Likewise, the graduation of fluid channel slope can correspond with a graduation in the height of protrusions forming the fluid channel (e.g. graduating both fluid channel slope and the height of protrusions on an infusion lumen in the distal direction). Alternatively, depending on the procedure, the angle can be left to shallow-out right near the opening to allow for a more linear directional exit of fluid out of the catheter tip. Optimizing vortical flow rates would help with further optimizing the reduction in whipping and migration in the catheter.

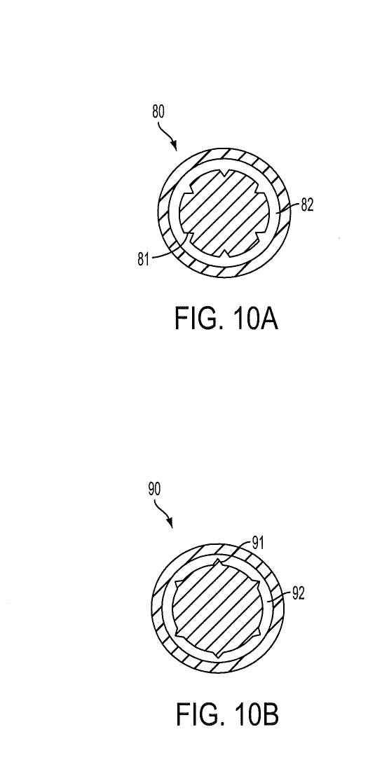

A method of manufacturing a catheter with rifles in the catheter lumen is also provided. Most catheters are manufactured using a process called extrusion, where a raw plastic or polymer material, typically in pellet form, is melted and formed into the shape that the user desires. In the case of a catheter, that shape would be generally tubular in form. As described above, the rifles necessary to create spiral flow through the catheter are created by either protrusions or notches in the catheter inner wall, and are typically present through substantially the entire length of the catheter lumen. The process of manufacturing a rifled catheter with catheter channels is done with the use of a die. The die is typically a metal structure with one or more openings that are shaped into the desired profile of the catheter. The molten polymer is pushed through the die and as it exits the die, it undergoes a cooling stage that causes the polymer to return to solid form and retain the shape of the die opening. FIG. 10A shows an example of a die 80 that will form protrusions in the catheter inner wall. The molten polymer is pushed through the opening 82, and will conform to and fill the shape of the entire opening 82, including the recessed areas 81 forming the protrusion. Spiraling of the protrusions can be created using a variety of methods. For example, as the molten polymer is being pushed through the opening 82, the inner section of the die can rotate at a speed commensurate with the desired angle of the spiral. Alternatively, as the finished end of the tube is being pulled through the cooling stage, the pulling mechanism can rotate in a similar fashion to form the spiral effect. Alternative geometries of dies can be used depending on the desired channel geometry. FIG. 10B shows an example of a die 90 for creating a rifled catheter having notches in the catheter inner wall. As the molten polymer is pushed through the opening 92, the notches in the die will be carved out by the projections 91 in the die. Alternative methods of forming the spiral may include RF tipping, including a mandrel matching the desired channeling pattern.

Polymers used to manufacture catheters according to the present invention can also have admixtures including fluoropolymer additives for an anti-thrombogenic surface. Admixtures such as those described in U.S. Pat. No. 8,603,070 and U.S. patent application Ser. No. 14,220,572, both to Lareau et al. and both incorporated herein by reference, can be used at the catheter material. Advantageously, since the anti-thrombogenic property of these additives is integral to the polymer admixture and is present throughout the bulk of the catheter shaft, the rifled channels can be formed with the instantaneous presence of an anti-thrombogenic surface, and without the difficult and additional step of trying to evenly coat the inner channeled walls with an anti-thrombogenic coating.

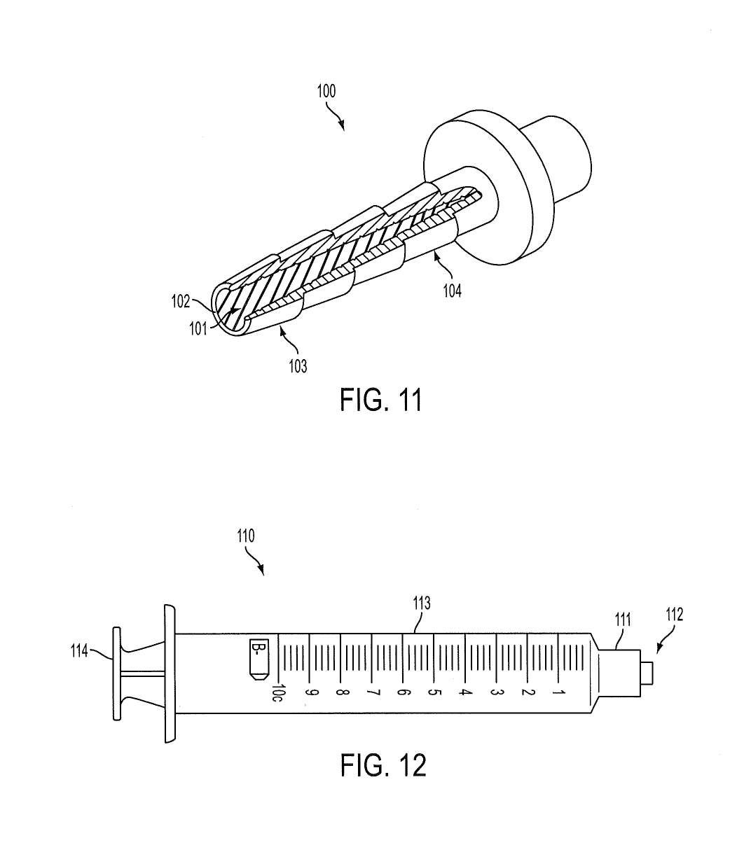

In an alternative embodiment, a fluid infusion system utilizes spiral flow of fluid introduced in vascular access components connected to the catheter. For example, implantable ports are used to provide fluid access to a part of the body, typically the vascular system, using a completely subcutaneous system. The port has a reservoir connected to a catheter via a port stem 100, an example of which is illustrated in FIG. 11. Fluids can be transferred between the port reservoir and the catheter via the lumen 101 in the port stem. A rifled catheter can be attached to the port stem 100 and secured by barbs or some other locking mechanism. In this embodiment, the port stem 100 connects with the proximal end of a catheter lumen. During infusion, the spiral flow will initiate at the proximal end 104 of the port stem 100, and continue to the proximal end 103 of the port stem as it enters the catheter. This is advantageous for the reasons provided above, and also to start the initiation of the vortical flow earlier in a port system where a port catheter is typically much shorter than other catheters, such as PICC catheters. Variations in the port stem spiral can implemented as described above with respect to the catheter spiral variations.

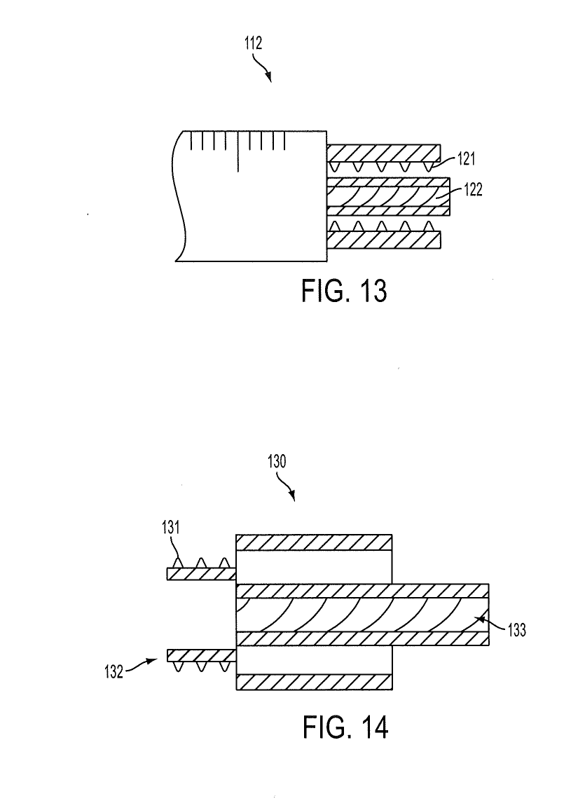

A fluid infusion system can also be created between a syringe 110 and catheter to introduce spiral flow early in the infusion process as shown in FIGS. 12-14. The syringe 110 of FIG. 12 has a plunger 114 for creating a positive or negative pressure in the fluid chamber 113. The system can include a syringe component 111 that would either attach to the distal end 112 of the syringe 110, or alternatively be an integral component to the syringe 110. An example of an integral component built into the distal end 112 of the syringe 110 is shown in FIG. 13. The lumen 122 at the distal end of the syringe is rifled to initiate the spiral flow. Threads 121 are included to connect the syringe to a luer of a vascular access product, such as a catheter described above. A conventional non-rifled syringe can also be used with an attachment 130 as shown in FIG. 14, so that a rifled lumen can be connected to the distal end 112 of the syringe 110. The syringe attachment includes a rifled insert lumen 133 and a proximal attachment member 132. The proximal attachment member 132 attaches to threads on the distal end of the syringe via attachment member threads 131. This system creates a spiral flow earlier in the injection process to promote a better vortical flow, increasing the stability of the catheter lumen during high flow rate and high pressure fluid infusion, reducing the whipping effect and the potential of catheter migration. Luers and extension tubes can also include rifled channels on inner walls for initiating vortical flow.

What is claimed is:

1. A catheter comprising:
a shaft comprising a proximal end, a distal end, an inner wall and a catheter longitudinal axis;
wherein, the inner wall comprises a substantially circular profile and a first catheter channel spiraling about the catheter longitudinal axis.

2. The catheter of claim 1, wherein the first catheter channel extends along the majority of the inner wall.

3. The catheter of claim 1, wherein the first catheter channel extends along substantially the entire inner wall.

4. The catheter of claim 1, wherein the first catheter channel is one of a plurality of channels spiraling about the catheter longitudinal axis.

5. The catheter of claim 4, wherein the plurality of channels is comprised of one or more protrusions on the inner wall.

6. The protrusions of claim 5, wherein the protrusions are one of triangular, rectangular, rounded and trapezoidal in shape.

7. The catheter of claim 4, wherein the plurality of channels is comprised of one or more notches in the inner wall.

8. The protrusions of claim 7, wherein the notches are one of triangular, rectangular, rounded and trapezoidal in shape.

9. An implantable port system comprising the catheter of claim 1, the implantable port system further comprising:
an implantable port comprising:
a reservoir,
a septum fluidly sealing an opening to the reservoir, and
an outlet stem in fluid communication with the reservoir,
wherein the outlet stem comprises a proximal end, a distal end, an outlet stem inner wall and an outlet stem longitudinal axis, and
wherein, the outlet stem inner wall comprises a substantially circular profile and an first outlet stem channel spiraling about the outlet stem longitudinal axis;
wherein the catheter is configured to connect to the outlet stem.

10. The outlet stem of claim 9, wherein the first outlet stem channel extends along substantially the entire outlet stem inner wall.

11. The outlet stem of claim 9, wherein the first outlet stem channel is one of a plurality of channels spiraling about the outlet stem longitudinal axis.

12. The implantable port system of claim 9, wherein the catheter longitudinal axis and the outlet stem longitudinal axis form a common axis when the catheter is connected to the outlet stem.

13. A catheter infusion system comprising the catheter of claim 1 further comprising a luer at the proximal end, the catheter infusion system further comprising;
a syringe comprising:
a syringe lumen comprising a proximal end and a distal end,
a plunger configured to flush the syringe lumen,
a syringe longitudinal axis, the inner wall comprising a substantially circular profile and a first syringe channel spiraling about the syringe longitudinal axis, and
a distal component, wherein the distal component comprises a distal component inner wall;
wherein the syringe is configured to connect to the luer.

14. The catheter infusion system of claim 13, wherein the distal component is configured to be attached to the syringe.

15. The catheter infusion system of claim 13, wherein the distal component is integral to the syringe.

16. The catheter infusion system of claim 13, wherein the first syringe channel extends along substantially the entire distal component.

17. The catheter infusion system of claim 13, wherein the catheter longitudinal axis and the syringe longitudinal axis form a common axis when the syringe is connected to the catheter.

18. The catheter of claim 1, wherein the first catheter channel comprises a helix disposed about the catheter longitudinal axis.

19. The catheter of claim 1, wherein the first catheter channel assumes a variable angle with the catheter longitudinal axis along a transition zone.

20. The catheter of claim 1 further comprising:
a second shaft comprising a proximal end, a distal end, an inner wall and a second catheter longitudinal axis;
wherein, the inner wall of the second shaft comprises a substantially circular profile and a second catheter channel spiraling about the second catheter longitudinal axis;
wherein a first shaft distal opening terminates distally of a second shaft distal opening;
wherein the first catheter channel comprises a protrusion on the inner wall of the first shaft, and the second catheter channel comprises a notch in the inner wall of the second shaft; and
wherein the first and second shafts are connected along at least a portion of their shafts.
